(19) United States
(12) Patent Application Publication
(10) Pub. No.: US 2022/0387482 A1
(43) Pub. Date: Dec. 8, 2022

(54) PERFORMANCE ENHANCEMENT DELIVERY SYSTEM

(71) Applicant: Michael Bland, Danapoint, CA (US)

(72) Inventor: Michael Bland, Danapoint, CA (US)

(73) Assignee: Michael Bland, Danapoint, CA (US)

(21) Appl. No.: 17/803,148

(22) Filed: Mar. 4, 2022

Related U.S. Application Data
(60) Provisional application No. 63/205,685, filed on Jan. 4, 2021.

(51) Int. Cl. A61K 33/00; A61K 31/122; A61K 31/198; A61P 3/02; A23L 33/105; A23L 33/155; A23L 33/175; A23L 33/16; A23L 33/125; A23P 10/30; A23P 10/40

(52) U.S. Cl. CPC A61K 33/00; A61K 31/122; A61K 31/198; A61P 3/02; A23L 33/105; A23L 33/155; A23L 33/175; A23L 33/16; A23L 33/125; A23P 10/30; A23P 10/40; A23V 2002/00

(57) ABSTRACT

The present disclosure provides an improvement of existing L-arginine containing supplements by providing both hydrogen crystals (H₂) and co-enzyme Q10 (Co-Q10) to produce a nutrient formulation, including compositions containing said formulation and methods of administering such formulation.

### CROSS REFERENCE TO RELATED APPLICATION

This application claims benefit under 35 U.S.C. § 119(e) to U.S. Provisional Application No. 63/205,268, filed on Jan. 4, 2021, which is hereby incorporated by reference in its entirety.

### FIELD OF THE INVENTION

The present invention relates generally to nutritional supplements, and more specifically to a non-toxic supplement which may be added to an existing product to achieve increased performance enhancement.

### BACKGROUND INFORMATION

Exercise performance has two components: endurance exercise capacity and muscle build-up/muscle strength. For endurance in exercise performance, it is critical to maintain a healthy heart and circulation, including efficient blood flow to the muscle, so that, for example, muscle tissue does not get sore while exercising.

For muscle build-up, which occurs primarily through protein formation in muscle tissue, the building-blocks for proteins are amino acids, which are used by the muscles through various metabolic pathways to form proteins (e.g., actin and myosin) and thus build muscle cells (myocytes) and fibers. Therefore, the supply of amino acids is critical for rapid and successful muscle build-up. Body builders have known this for a very long time, and many (if not most) have been using amino acid supplementation to ensure bioavailability. For example, L-arginine is a critical amino acid in this context, although a non-essential amino acid (meaning that it can be synthesized in vivo), as its supply becomes important where protein biosynthesis occurs at a high pace.

L-Arginine is a crystalline, free-form amino acid. It is an important factor in muscle metabolism and works to transport, store and excrete nitrogen and is involved in DNA synthesis. It is also a precursor of guanidophosphate, phosphoarginine, and creatine, three high-energy compounds found in muscles.

Arginine is very popular among athletes and bodybuilders because it is required in muscle metabolism—maintaining the nitrogen balance, and helping with weight control since it facilitates the increase of muscle mass, while reducing body fat.

Arginine is abundant in protamines and histones—both proteins associated with nucleic acids and was first isolated in 1895 from animal horn. Newborns may produce this amino acid too slowly and for them arginine should be seen as an essential amino acid.

Arginine is extremely useful in enhancing the immune system, and it increases the size and activity of the thymus gland, which is responsible for manufacturing T lymphocytes—the much talked about T-cells, which assist the immune system. For this reason, arginine might be an important nutrient for people suffering from AIDS and other malignant diseases which suppress the immune system.

In the pancreas arginine is used to release insulin and in the pituitary gland it is a component of human growth hormone, and is used in sexual stimulants, as people report longer and more intense orgasms when their intake of arginine is increased.

Arginine is also important in liver health and assists in neutralizing ammonia in the liver, while it is also involved in the skin and connective tissue—making it important in healing and repair of tissue as well as the formation of collagen and building of new bone and tendons.

Arginine is found in seminal fluid and L-arginine is used in the treatment of male sexual health and has been used in the treatment of sterility.

While supplements containing L-arginine are on the market, improvements are required to increase bioavailability of said amino acid.

### SUMMARY OF THE INVENTION

The present invention provides an improvement of the existing L-arginine containing supplements by providing both hydrogen crystals (H₂) and co-enzyme Q10 (Co-Q10).

In embodiments, a composition is disclosed including co-enzyme Q10, hydrogen crystals, carbohydrates, vitamin C, vitamin D₃, vitamin K, vitamin B₆, folic acid, vitamin B₁₂, Mg, L-arginine, L-citrulline, red wine extract, a ginseng mix, and a mineral mix.

In one aspect, the ginseng mix includes Panax ginseng extract, Panax notoginseng extract, Rosa roxburghii extract, saponin, polyphenols, vitamin C, ginsenodide R1, and astragaloside.

In a related aspect, the mineral mix comprises Al, Sb, Ba, Be, Bi, B, Br, Cd, C, Ce, Cl, Cr, Co, Cu, Dy, Er, Eu, F, Gd, Ge, Au, Hf, Ho, In, I, Ir, Fe, La, Li, Lu, Mg, Mn, Mo, Nd, Ni, Nb, Os, Pd, P, Pt, K, Pr, Re, Rh, Rb, Ru, Sm, Sc, Se, Si, Ag, Na, Sr, S, Ta, Te, Tb, Tl, Th, Tm, Sn, Ti, W, V, Yb, Y, An, Zr, and fluvic acid.

In one aspect, one serving of the composition comprises between about 50 mg to about 54 mg of co-enzyme Q10, between about 80 mg to about 90 mg of hydrogen crystals.

In another aspect, the composition further includes between about 4 g to about 5 g of carbohydrates, between about 60 mg to about 76 mg of vitamin C, between about 2500IU to 3155IU of vitamin D₃, between about 20 μg to about 25 μg vitamin K, between about 2 mg to about 3 mg of vitamin B₆, between about 400 mg to about 505 mg of folic acid, between about 6 μg and 8 μg of vitamin B₁₂, between about 20 mg to about 25 mg of Mg, between about 5110 mg and 6450 mg of L-arginine, between about 1010 mg to about 1275 mg of L-citrulline, between about 100 mg to about 126 mg of red wine extract, between about 50 mg to about 63 mg of ginseng mix; and between about 100 mg to about 126 mg of mineral mix.

In a related aspect, the composition further contains one or more additional components including a carrier, an excipient, a binder, a colorant, a flavoring agent, a preservative, a buffer, a dilutant and combinations thereof.

In one aspect, the composition is a human dietary supplement.

In another aspect, the composition is in a dosage form including a capsule, a cachet, a pill, a tablet, a powder, a granule, a pellet, a bead, a particle, a troche, and a lozenge.

In a related aspect, the one or more additional components is a carrier. In a further related aspect, the one or more additional components is an excipient.

In another aspect, the one or more additional components is a binder.

In one aspect, the one or more additional components is a colorant. In a related aspect, the one or more additional components is a flavoring agent.

In another aspect, the one or more additional components is a preservative.

In one aspect, the one or more additional components is a buffer.

In a related aspect, the one or more additional components is a dilutant.

In embodiments, a composition is disclosed including co-enzyme Q10, hydrogen crystals, carbohydrates, vitamin C, vitamin D₃, vitamin K, vitamin B₆, folic acid, vitamin B₁₂, Mg, L-arginine, L-citrulline, red wine extract, a ginseng mix, a mineral mix and one or more additional components including a carrier, an excipient, a binder, a colorant, a flavoring agent, a preservative, a buffer, and a dilutant, where the compositions is in a dosage form including a capsule, a cachet, a pill, a tablet, a powder, a granule, a pellet, a bead, a particle, a troche, and a lozenge.

In one aspect, the dosage form is a capsule.

In another aspect, the dosage form is a cachet.

In one aspect, the dosage form is a powder.

In one aspect, the combined product may be used as a medical preventative and/or performance enhancer.

In another aspect, the combined product may be effectively used to assist in neutralizing homocycteine to reduce plaque in arteries. In a related aspect, the product may cross the blood brain barrier to induce secretion of pituitary hormones, including but not limited to, growth hormone. In a further related aspect, the combined product may be used to increase testosterone levels in a subject in need thereof.

In one aspect, the combined product increases mobility of arginine into the blood stream and/or increases bioavailability of the amino acid. In a related aspect, the combined product aids in the prevention of excessive oxidative stress and aids in maintaining antioxidant levels in the subject, thereby reducing fatigue, soreness and at the same time increasing the speed of recovery from exercise.

In another aspect, the combined product helps to maintain homeostasis in a subject without synthetic toxic side effects.

### BRIEF DESCRIPTION OF THE DRAWINGS

FIG. 1 is a graph showing results of a study using the composition

### DETAILED DESCRIPTION OF THE INVENTION

Before the present composition, methods and methodologies are described, it is to be understood that this invention is not limited to particular compositions, methods, and experimental conditions described, as such compositions, methods, and conditions may vary. It is also to be understood that the terminology used herein is for the purpose of describing particular embodiments only, and is not intended to be limiting, since the scope of the present invention will be limited only by the appended claims.

As used in this specification and appended claims, the singular forms “a,” “an,” and “the” include plural references unless the context clearly dictates otherwise. Thus, for example, references to “amino acid” includes one or more amino acids, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and material similar or equivalent to those described herein can be used in the practice of testing of the invention, as it will be understood that modifications and variations are encompassed within the spirit and scope of the instant disclosure.

As used herein, “about,” “approximately,” “substantially,” and “significantly” will be understood by a person of ordinary skill in the art and will vary in some extent depending on the context in which they are used. If there are uses of the term which are not clear to persons of ordinary skill in the art given the context in which it is used, “about” and “approximately” will mean plus or minus <10% of the particular term and “substantially” and “significantly” will mean plus or minus >10% of the particular term. In embodiments, compositions may “contain,” “comprise,” or “consist essentially of” a particular component or group of components, where the skilled artisan would understand the latter to mean the scope of the claim is limited to the specified material or steps “and those that do not materially affect the basic and novel characteristic(s)” of the claimed invention.

As shown below, Co-Q10 has the following molecular structure:

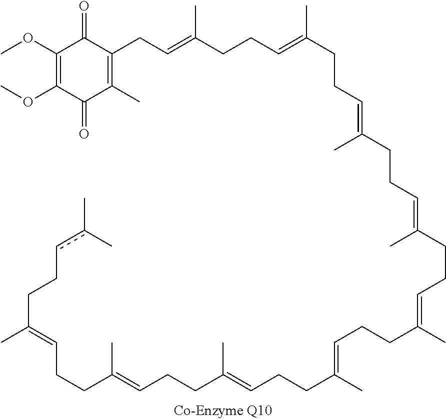

As described above, L-arginine stimulates growth hormone release. While not being bound by theory, by keeping blood L-arginine levels high for an extended period of time, the combined composition as envisaged, allows for penetration across the blood brain barrier (BBB) and stimulates the pituitary to produce growth hormone, which thereby stimulates muscle build-up and aids in maintaining circulation and modulating homeostasis. The hydrogen component enhances the L-arginine penetration.

Arginine is characterized as a nonessential amino acid. Specifically, arginine can be independently manufactured by the human body, and does not need to be obtained directly through dietary intake. Arginine plays a significant role in healing, cell division, immune function, the elimination of ammonia from the body and the release of hormones. Arginine is presently used in the dietary supplement industry to supplement Arginine production in the body. Arginine is also presently used in the dietary supplement industry to boost human growth hormone (HGH) production, increase vasodilation, enhance blood circulation, increase oxygen flow to the muscles, and boost nitric oxide (NO) production. Various supplemental arginine forms are available in the consumer marketplace.

The vasodilating effect of ingested arginine takes considerable time to manifest since arginine requires extensive metabolism to yield NO. Additionally, considerable amounts of arginine are required to produce a significant vasodilating effect, with common doses ranging from eight to twenty-four grams per day.

Citrulline is an alpha-amino acid naturally occurring in the human body, and does not need to be obtained directly through dietary intake. In vivo, citrulline is made from the amino acid ornithine, along with carbamoyl phosphate in one of the central reactions in the urea cycle. Citrulline is also produced during the metabolism of arginine in the body. Citrulline is presently used in the dietary supplement industry to supplement citrulline production in the body. By itself, citrulline has no vasodilating properties. Citrulline is also water insoluble, which reduces its bioavailability and limits the forms in which citrulline may be effectively used.

By taking one scoop every 12 hours consisting of 15 to 20 grams of L-arginine will also cross the BBB and induce secretion of growth hormone, resulting in an increase in testosterone. This may be verified by measuring testosterone levels before and after subject ingests the formulation.

In embodiments, the composition contains a combination of co-enzyme Q10, hydrogen crystals, carbohydrates, vitamin C, vitamin D₃, vitamin K, vitamin B₆, folic acid, vitamin B₁₂, Mg, L-arginine, L-citrulline, red wine extract, ginseng mix (i.e., Panax ginseng extract, Panax notoginseng extract, Rosa roxburghii extract, saponin, polyphenols, vitamin C, ginsenodide R1, and astragaloside; e.g., similar to ASTRAGIN®, available from NuLivScience, Brea, Calif.), and a mineral mix comprising the elements Al, Sb, Ba, Be, Bi, B, Br, Cd, C, Ce, Cl, Cr, Co, Cu, Dy, Er, Eu, F, Gd, Ge, Au, Hf, Ho, In, I, Ir, Fe, La, Li, Lu, Mg, Mn, Mo, Nd, Ni, Nb, Os, Pd, P, Pt, K, Pr, Re, Rh, Rb, Ru, Sm, Sc, Se, Si, Ag, Na, Sr, S, Ta, Te, Tb, Tl, Th, Tm, Sn, Ti, W, V, Yb, Y, An, Zr, and fluvic acid. In a related aspect, one serving of the composition (about 13 g to about 16 g) comprises between about 50 mg to about 54 mg co-enzyme Q10, between about 80 mg to about 90 mg of hydrogen crystals (equivalent to one hydrogen tablet, e.g., DRINKHRW® Rejuvenation, Oxnard, Calif.); between about 4 g to about 5 g of carbohydrates, between about 60 mg to about 76 mg of vitamin C, between about 2500IU to 3155IU of vitamin D₃, between about 20 μg to about 25 μg vitamin K, between about 2 mg to about 3 mg of vitamin B₆, between about 400 mg to about 505 mg of folic acid, between about 6 μg and 8 μg of vitamin B₁₂, between about 20 mg to about 25 mg of Mg, between about 5110 mg and 6450 mg of L-arginine, between about 1010 mg to about 1275 mg of L-citrulline, between about 100 mg to about 126 mg of red wine extract, between about 50 mg to about 63 mg of ginseng mix; and between about 100 mg to about 126 mg of mineral mix.

Red wine (RW) extract is obtained after the fermentation process of grapes has been completed, which RW extract contains nutrients from the flesh, skin and seeds (may or may not have seeds, depending on the grapes) of grapes that have been fermented.

The compositions and formulations according to the present invention may further comprise one or more acceptable carriers or excipients. Acceptable carriers and excipients may be combined with (e.g., mixed with or coated with) the composition as disclosed herein. Carriers/excipients include fillers or extenders, binders, wetting agents, emulsifiers, and anti-caking agents.

Additionally, or alternatively, the carrier may be any of the one or more additives disclosed herein, wherein the contemplated composition is in powder form for any of the disclosed additives. As such, the additive may be used as the “platform” for the composition. In addition to the additives disclosed herein, additional carriers also include a polysaccharide complex. Examples of a polysaccharide complex include highly branched galactose. Galactose may be highly branched with rhamnose, arabinose, and/or glucuronic acid.

A wide number of acceptable carriers are well known in the art. The carrier need only be suitable for administration to a human and be able to act as a carrier without substantially affecting the desired activity of the composition. Also, the carrier(s) may be selected based on the desired administration route and dosage of the composition or formulation. For example, the compositions according to the present invention are suitable for are suitable for use on a variety of dosage forms such as liquid form and solid form (e.g., a chewable bar or wafer). In desirable embodiments as discussed below, the composition or formulation comprises a solid dosage form such as a tablet, caplet or capsule, lozenges, and chewing gum. Examples of suitable carriers for use in tablet, caplet and capsule compositions or formulations include, but are not limited to, inert organic and inert inorganic carrier materials such as gelatin, starch, magnesium stearate, talc, gums, silicon dioxide, stearic acid, cellulose and the like. Desirably, the carrier is substantially inert. In embodiments, the composition or formulation is tablet, caplet, capsule or lozenge which uses pharmaceutically acceptable controlled release, time release or extended release technology; suitable technologies are known in the art.

Advantageously, one or more of the exemplary compositions described herein may be used as a composition, either alone or part of a more complex composition containing any number of additional ingredients. It will be readily apparent to those skilled in the art which specific ingredients may be beneficially included in such compositions. Furthermore, any one or more of the exemplary compositions disclosed herein may be administered in any dosage form common in the art. For example, the compositions disclosed herein may be administered in the form of a powder to be mixed in a liquid, or a solid dosage form such as a tablet, caplet, capsule, a ready-to-mix (e.g., ready-to-drink (RTD)) formulation, lozenges, nutritional bars, food bars or candy bars. More specifically, the contemplated composition may be an effective dosage of the composition as disclosed herein is formulated as a powder to be mixed in a liquid or in a solid dosage form to be formed as one or more tablets, one or more caplets, one or more capsules, one or more lozenges, one or more nutritional bars, one or more food bars, or one or more candy bars.

In embodiments, the contemplated compositions disclosed herein may further include one or more pharmaceutically acceptable excipients or carriers. In a related aspect, the composition may be in the form of a powdered dietary supplement (e.g., ready-to-mix or RTD powder), capsule, tablet, caplet, or lozenge. In a further related aspect, the compositions may be formulated (e.g., agglomerated) to be shelf stable for six, twelve, eighteen, twenty, twenty-four, thirty, thirty-six, forty, forty-two, forty-eight months or longer.

In embodiments, the contemplated composition is a nutraceutical. In a related aspect, the composition is a dietary ingredient and/or a dietary supplement. The composition may be a medical food or drug. In a related aspect, the composition is designated generally recognized as safe (GRAS) or as a new dietary ingredient (NDI).

As used herein, the term “nutraceutical” and “nutraceutically acceptable” are used to refer to any substance that is food or part of a food and provides medical and/or health benefits, including prevention and/or treatment of disease. Hence, compositions falling under the label of “nutraceutical” or “nutraceutically acceptable” may range from isolated nutrients, nutritional or dietary ingredients or dietary supplements and specific diets to genetically engineered designer foods, herbal products and processed foods such as beverages. In a more technical sense, the term has been used to refer to a product isolated or purified from foods, and generally sold in medicinal forms not associated with foods and demonstrated to have a physiological benefit or protection from chronic disease. The effect of the combined product as disclosed herein shows that the product supports the action of testosterone, which hormone promotes muscle development and build up. In addition, male sexual function may be enhanced as a result. Further, by aiding circulatory function, the action of the combined product as claimed aids in endurance during exercise. In one aspect, the combined product as claimed herein may be effective in increasing performance of athletes.

In a related aspect, the combined product as disclosed herein does not produce harmful side effects, while aiding in healthy heart effects.

In one aspect, the combined product helps maintain muscle mass as a subject grows older, as maintaining muscle mass is critical for overall fitness, coordination and hormonal balance as a subject ages. Further, as lack of growth hormone is associated with heart disease, and lack of testosterone is associated with erectile dysfunction, the combined product as disclosed herein helps with both conditions, including relieving the mental stress that may be associated with the latter.

In embodiments, the formulation of the combined product comprises H₂, Co-Q10, vitamin C, vitamin D₃, vitamin K, vitamin B₆, folate, vitamin B₁₂, magnesium, L-arginine, L-citrulline, red wine extract, a ginseng extract mix, and a trace mineral mix.

### EXAMPLES

### Example 1

### Study

A study was carried out to analyze various metrics to determine the efficacy of the supplement.

No of Participants: 6

Ages: 34 yr.o (male: Subject A); 40 yr.o (male: Subject B); 45 yr.o (male: Subject C); 56 yr.o (female: Subject D); 40 yr.o (female: Subject E); 50 yr.o (female: Subject F).

Subject A XXL13 g/day (XXL+Co-Q10+hydrogen, 1 Serving) for 1 month every morning before work out; Subject B L-arginine (XXL−Co-Q10, −hydrogen) 13 g/day (1 Serving) for six (6) weeks every morning before workout; Subject C (placebo: natural yuzu flavor; natural pink lemonade; sweter; xylitol; citric acid, beet juice powder; organic stevia; natural flavor for strawberry powder; calcium silicate; and trace mineral bulk-powder) 13 g/day (1 Serving) for five (5) weeks every morning before workout; Subject D XXL (XXL+Co-Q10+hydrogen, 1 Serving) 13 g/day for seven (7) weeks every morning before workout; Subject E L-arginine (XXL−Co-Q10, −hydrogen) 13 g/day (1 Serving) for five (5) weeks every morning before workout; Subject F (placebo) 13 g/day (1 Serving) for four (4) weeks every morning before workout.

Subjects worked out a local gym, performing their normal routines (e.g., treadmill, free-weights, including using other fitness machines).

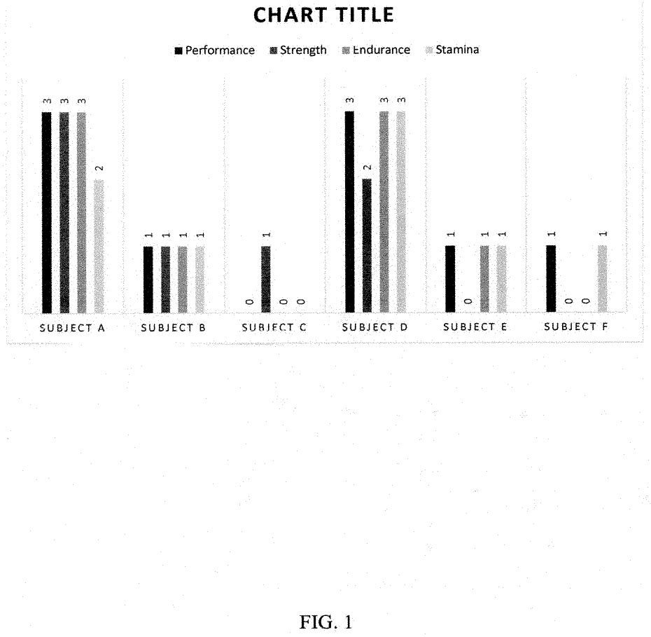

Participants used the following rating system to capture their personal observations to determine the effects of the supplement on their workouts: Performance (None=0 pts; Fair=1 pt; Good=2 pts; Excellent=3 pts); Strength (same ratings); Endurance (same ratings); Stamina (same ratings). The results are shown in Table 1 and FIG. 1.

TABLE 1
Results of Study

| | Performance | Strength | Endurance | Stamina |
|---|---|---|---|---|
| Subject A | 3 | 3 | 3 | 2 |
| Subject B | 1 | 1 | 1 | 1 |
| Subject C | 0 | 1 | 0 | 0 |
| Subject D | 3 | 2 | 3 | 3 |
| Subject E | 1 | 0 | 1 | 1 |
| Subject F | 1 | 0 | 0 | 1 |

As may be seen in Table 1 (and graphically in FIG.1), XXL+Co-Q10+hydrogen was observed to be the superior product across all metrics tested.

Although the invention has been described in the above example, it will be understood that modifications and variations are encompassed within the spirit and scope of the invention. Accordingly, the invention is limited only by the following claims.

I claim:

1. A composition comprising co-enzyme Q10, hydrogen crystals, carbohydrates, vitamin C, vitamin D₃, vitamin K, vitamin B₆, folic acid, vitamin B₁₂, Mg, L-arginine, L-citrulline, red wine extract, a ginseng mix, and a mineral mix.

2. The composition of claim 1, wherein the ginseng mix comprises Panax ginseng extract, Panax notoginseng extract, Rosa roxburghii extract, saponin, polyphenols, vitamin C, ginsenodide R1, and astragaloside.

3. The composition of claim 1, wherein the mineral mix comprises Al, Sb, Ba, Be, Bi, B, Br, Cd, C, Ce, Cl, Cr, Co, Cu, Dy, Er, Eu, F, Gd, Ge, Au, Hf, Ho, In, I, Ir, Fe, La, Li, Lu, Mg, Mn, Mo, Nd, Ni, Nb, Os, Pd, P, Pt, K, Pr, Re, Rh, Rb, Ru, Sm, Sc, Se, Si, Ag, Na, Sr, S, Ta, Te, Tb, Tl, Th, Tm, Sn, Ti, W, V, Yb, Y, An, Zr, and fluvic acid.

4. The composition of claim 1, wherein one serving of the composition comprises between about 50 mg to about 54 mg co-enzyme Q10, between about 80 mg to about 90 mg of hydrogen crystals.

5. The composition of claim 4, further comprising between about 4 g to about 5 g of carbohydrates, between about 60 mg to about 76 mg of vitamin C, between about 2500IU to 3155IU of vitamin D₃, between about 20 μg to about 25 μg vitamin K, between about 2 mg to about 3 mg of vitamin B₆, between about 400 mg to about 505 mg of folic acid, between about 6 μg and 8 μg of vitamin B₁₂, between about 20 mg to about 25 mg of Mg, between about 5110 mg and 6450 mg of L-arginine, between about 1010 mg to about 1275 mg of L-citrulline, between about 100 mg to about 126 mg of red wine extract, between about 50 mg to about 63 mg of ginseng mix; and between about 100 mg to about 126 mg of mineral mix.

6. The composition of claim 5, wherein the composition further comprises one or more additional components selected from the group consisting of a carrier, an excipient, a binder, a colorant, a flavoring agent, a preservative, a buffer, a dilutant and combinations thereof.

7. The composition of claim 1, wherein the composition is a human dietary supplement.

8. The composition of claim 1, wherein the composition is in a dosage form selected from the group consisting of a capsule, a cachet, a pill, a tablet, a powder, a granule, a pellet, a bead, a particle, a troche, and a lozenge.

9. The composition of claim 1, wherein the one or more additional components is a carrier.

10. The composition of claim 1, wherein the one or more additional components is an excipient.

11. The composition of claim 1, wherein the one or more additional components is a binder.

12. The composition of claim 1, wherein the one or more additional components is a colorant.

13. The composition of claim 1, wherein the one or more additional components is a flavoring agent.

14. The composition of claim 1, wherein the one or more additional components is a preservative.

15. The composition of claim 1, wherein the one or more additional components is a buffer.

16. The composition of claim 1, wherein the one or more additional components is a dilutant.

17. A composition comprising: co-enzyme Q, hydrogen crystals, carbohydrates, vitamin C, vitamin D3, vitamin K, vitamin B₆, folic acid, vitamin B₁₂, Mg, L-arginine, L-citrulline, red wine extract, a ginseng mix, a mineral mix and one or more additional components selected from the group consisting of a carrier, an excipient, a binder, a colorant, a flavoring agent, a preservative, a buffer, and a dilutant, wherein the compositions is in a dosage form selected from the group consisting of a capsule, a cachet, a pill, a tablet, a powder, a granule, a pellet, a bead, a particle, a troche, and a lozenge.

18. The composition of claim 12, wherein the dosage form is a capsule.

19. The composition of claim 12, wherein the dosage form is a cachet.

20. The composition of claim 12, wherein the dosage form is a powder.
